(19) United States
(12) Patent Application Publication
(10) Pub. No.: US 2017/0189703 A1
(43) Pub. Date: Jul. 6, 2017

(54) RADIO FREQUENCY TREATMENT APPARATUS WITH SELF-ADAPTIVE TREATMENT DEPTH

(71) Applicant: Shenzhen Peninsula Medical Co., Ltd., Shenzhen (CN)

(72) Inventor: XIAOBING LEI, Shenzhen (CN)

(73) Assignee: Shenzhen Peninsula Medical Co., Ltd., Shenzhen (CN)

(21) Appl. No.: 15/466,620

(22) Filed: Mar. 22, 2017

(30) Foreign Application Priority Data
Sep. 9, 2016 (CN) 201610812337.1
Sep. 9, 2016 (CN) 201610816116.1

(51) Int. Cl. A61N 1/40; A61N 1/06

(52) U.S. Cl. CPC A61N 1/40; A61N 1/06

(57) ABSTRACT

A bipolar radiofrequency (RF) treatment apparatus includes: a plurality of bipolar RF treatment devices each including an RF energy generator, and a first RF electrode and a second RF electrode uniquely connected to the RF energy generator. The first and second RF electrodes are arranged on a same treatment surface in a same order and in a reverse order, respectively. By switching polarities of the first RF electrode and the second RF electrode connected to the RF energy generator, a distribution of electrical currents between the first and second RF electrodes based on mutual attraction and/or repulsion between magnetic fields associated with the electrical currents can be adjusted, thereby realizing control of a treatment depth and treatment current density. A magnetic field generating mechanism can be included to further affect the current distribution.

### CROSS-REFERENCE TO RELATED APPLICATIONS

The present application claims priority to Chinese Patent Application No. CN 201610812337.1 filed on Sep. 9, 2016, and Chinese Patent Application No. CN 201610816116.1 filed on Sep. 9, 2016. The disclosures of these applications are hereby incorporated by reference in their entirety.

### BACKGROUND

In existing low-frequency radio frequency (RF) treatment, the RF penetration depth is uncontrollable, making the energy concentrated in the epidermis and dermis. As such, subcutaneous energy accumulation is too low for the dermis deep-level tightening or subcutaneous fat layer shaping, resulting in poor treatment results. The need for a long time cumulative treatment leads to a very slow treatment speed.

Skin wrinkles and other skin aging symptoms mainly result from the loss of dermal collagen. Currently, treatment methods may include using thermal stimulation of collagen to promote the renewal of collagen and related treatment of symptoms. For example, laser, radio frequency and other physical technologies can be adopted in treatment equipment. As the RF has non-invasive characteristics, it is widely adopted by customers. At present, in non-invasive radiofrequency treatment, the heating level is relatively shallow, and thus cannot be a good solution to the deep skin condition treatment, and the treatment effects after treatment usually maintain only a short period of time. Some methods adopt cooling of epidermis, while implementing a high-dose radiofrequency treatment. Some other methods adopt the use of invasive treatment, such as the radio frequency electrode made into a microneedle, that enters subcutaneous region to deliver radiofrequency energy. These methods not only seriously increase the cost of equipment, while the skin damage is serious, and can cause uncomfortable situation for the patients.

In the field of low frequency radiofrequency treatment, especially its output frequency is mainly below 20 MHz frequency, the heating mechanism is mainly impedance heating. The energy between the heating tissues Q=I²*R*t (where I is the current density, R is the impedance of the body tissue, and t is the duration of the current flow). As the RF current flows in the direction of the smallest impedance, the current is mainly concentrated in the epidermal and dermal interface, as the impedance of this region is similar to that of the dermis layer, but far below the epidermis impedance.

### SUMMARY

In view of the above-mentioned drawbacks of the prior art, it is an object of the present invention to provide a RF therapeutic apparatus having a therapeutic apparatus having a depth self-regulating function, thereby solving a problem that the treatment depth is uncontrollable in the conventional low-frequency RF treatment apparatus, and the resulting poor treatment effects and long treatment time.

It is another object of the present invention to improve the treatment depth under low frequency radiofrequency treatment to improve the accuracy of treatment and treatment efficiency.

In order to achieve the above objects and other related objects, the present disclosure provides the following technical solutions.

A bipolar radiofrequency treatment apparatus comprising a plurality of bipolar radiofrequency treatment devices, each of the bipolar radiofrequency treatment devices comprising a radio frequency energy generator, and first and second radio frequency electrodes uniquely connected to the radio frequency energy generator. The first radio frequency electrode and the second radio frequency electrode being arranged on the same treatment surface in sequence and in reverse order, respectively. Each of the radio frequency energy generators is configured to switch an electrode polarity to be applied to the first and second radio frequency electrodes based on predetermined specifications.

in some embodiments, each of the first radio frequency electrodes has a first equal interval on the therapeutic surface, and each of the second radio frequency electrodes has a second equal interval on the therapeutic surface.

In some embodiments, the first radio frequency electrodes and the second radio frequency electrodes have rectangular shapes.

In some embodiments, the first radio frequency electrodes and the second radio frequency electrodes have ring shapes.

In some embodiments, each of the radio frequency energy generators is configured to control a current amplitude and a frequency applied to the first radio frequency electrodes and the second radio frequency electrodes.

In some embodiments, each of the radio frequency energy generators is configured to control a current amplitude and a frequency applied to the first radio frequency electrode and the second radio frequency electrode.

In some embodiments, each of the radio frequency energy generators is configured to apply a current with a frequency of no more than 20 MHz to the first radio frequency electrode and the second radio frequency electrode.

Embodiments disclosed herein can have one or more of the following advantages: by controlling the polarities of the two electrodes of each bipolar RF treatment device, the direction of the RF current can be controlled, the RF current in the subcutaneous distribution, such as a degree of sparseness, and a penetration depth, can be improved, to thereby achieve the depth of radiofrequency treatment being self-regulated, facilitating focused treatment of some of the symptoms, and enhancing the therapeutic effect and treatment efficiency.

In another aspect, a bipolar radiofrequency treatment device is provided with adjustable depth of treatment, the device including: at least one or more radio frequency generators each forming a set of bipolar radio frequency unit by uniquely connecting a first radio frequency electrode and a second radio frequency electrode. Each of the first radio frequency electrodes and each of the second radio frequency electrodes being arranged in the same order and in reverse order, respectively, to form a therapeutic surface in the same direction. Between a first radio frequency electrode and/or a second radio frequency electrode for the same or/and different radio frequency generators, at least one magnetic field generating mechanism is provided.

In some embodiments, there is provided one radio frequency generator in which at least one magnetic field generating mechanism is provided on a therapeutic surface between the first radio frequency electrode and the second radio frequency electrode on the radio frequency generator.

In some embodiments, there are provided multiple radio frequency generators in which at least one magnetic field generating mechanism is provided on a therapeutic surface between the first radio frequency electrodes and between the second radio frequency electrodes or/and between the first radio frequency electrode and the second radio frequency electrode.

In some embodiments, each of the magnetic field generating mechanisms being spaced apart on the therapeutic surface.

In some embodiments, the magnetic field generating mechanism includes an electromagnetic coil and a power supply control module for applying different current directions or/and different current amplitudes to the electromagnetic coil according to the setting requirements.

In some embodiments, the magnetic field generating mechanism is a magnet.

In some embodiments, the radio frequency generator is a low-frequency generator.

In some embodiments, the first radio frequency electrodes have a first equal interval on the therapeutic surface and the second radio frequency electrodes have a second equal interval on the therapeutic surface.

In some embodiments, the first radio frequency electrodes and the second radio frequency electrodes are rectangular radio frequency electrodes, respectively.

In some embodiments, the first radio frequency electrodes and the second radio frequency electrodes are annular radio frequency electrodes, respectively.

In view of the above, various embodiments disclosed herein can have the following beneficial effects: the embodiments provide a magnetic field generating mechanism on the treatment surface to generate a magnetic field, and the effect of the magnetic field mutual attraction and mutual repulsion is used to improve the density and the degree of aggregation of the subcutaneous radio frequency current so as to control the subcutaneous radio frequency energy penetration and aggregation, in order to achieve accurate treatment of the treatment area, thereby shortening the treatment time and improve the treatment efficiency.

### BRIEF DESCRIPTION OF DRAWINGS

To more clearly illustrate the embodiments of the disclosure, the following is a brief description of the drawings, which are for illustrative purpose only. For those of ordinary skills in the art, other drawings of other embodiments can become apparent based on these drawings.

FIG. 1 is a schematic diagram of a bipolar RF treatment apparatus with a treatment depth self-regulation function;

FIG. 2 illustrates a distribution of RF current and magnetic field when only one bipolar treatment device is present;

FIG. 3A illustrates a distribution of magnetic field when only two bipolar treatment devices are present with RF electric currents of a same direction;

FIG. 3B illustrates a distribution of magnetic field when only two bipolar treatment devices are present with RF electric currents of different directions;

FIG. 4 is a schematic diagram of a bipolar RF treatment apparatus with a treatment depth self-regulation function according to some embodiments;

FIG. 5 illustrates a distribution of magnetic field for three RF electric currents of a same direction;

FIG. 6 is a schematic diagram of an apparatus with three RF electric currents of different directions;

FIG. 7 illustrates a distribution of magnetic field for the apparatus of FIG. 6;

FIG. 8 is a schematic diagram of an apparatus with three RF electric currents of different directions according to some other embodiments;

FIG. 9 illustrates a distribution of magnetic field for the apparatus of FIG. 8;

FIG. 10 is a schematic diagram of a controller according to some embodiments;

FIG. 11 is a schematic diagram of an RF electrode according to some embodiments;

FIG. 12 is a schematic diagram of an RF electrode according to some other embodiments;

FIG. 13 is a schematic diagram of RF current distribution of a bipolar RF treatment apparatus with adjustable treatment depth having one magnetic field;

FIG. 14 is a schematic diagram of RF current distribution of a bipolar RF treatment apparatus with adjustable treatment depth having two independent magnetic fields;

FIG. 15 is a schematic diagram of RF current distribution of a bipolar RF treatment apparatus with adjustable treatment depth having multiple independent RF electrode pairs with a set of magnetic fields;

FIG. 16 is a schematic diagram of RF current distribution of a bipolar RF treatment apparatus with adjustable treatment depth having multiple independent RF electrode pairs with multiple sets of magnetic fields; and

FIG. 17 is a schematic diagram illustrating a principle of a magnetic field generating mechanism according to some embodiments.

### DETAILED DESCRIPTION

In the following, with reference to the drawings of various embodiments disclosed herein, the technical solutions of the embodiments of the disclosure will be described in a clear and fully understandable way. It is obvious that the described embodiments are merely a portion but not all of the embodiments of the disclosure. Based on the described embodiments of the disclosure, those ordinarily skilled in the art can obtain other embodiment(s), which come(s) within the scope sought for protection by the disclosure.

It should be noted that structures, proportions, sizes, etc. shown in the drawings shown in the drawings are intended to cover the same information as those set forth in the specification and are not to be construed, as being understood to those skilled in the art, to be limiting the invention. Modification of any structure, change of the proportionality or adjustment of the size, shall remain within the scope of the present invention without affecting the efficacy and achievable effect of various embodiments disclosed herein. The terms quoted in the present specification, such as “up,” “down,” “left,” “right,” “middle,” and “one,” are merely for illustrative purposes. The change or adjustment of its relative relationship, when not substantially changing the technical content, is also considered as within the scope of the invention.

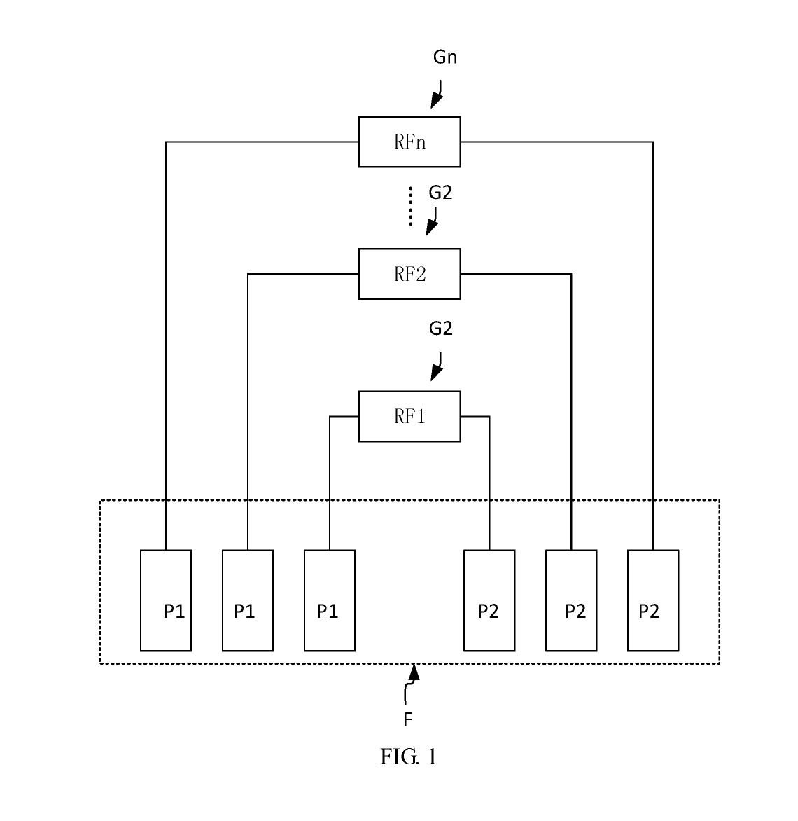

Referring to FIG. 1, some embodiments disclosed herein provide a bipolar radiofrequency treatment device having a therapeutic depth self-regulating function comprising a plurality of bipolar radiofrequency treatment devices or units (G1, G2 . . . Gn), each of the bipolar radiofrequency treatment devices comprising a RF energy generator (RF1, RF2 . . . RFn), and a first RF electrode P1 and a second radio frequency electrode P2, which are uniquely connected to the RF energy generator, each of the radio frequency energy generators is capable of switching polarity on the first radio frequency electrode and the second radio frequency electrode connected thereto, and each of said first radio frequency electrodes and each of said second radio frequency electrodes are arranged on the same treatment surface F in the same order and in reverse order, respectively.

The above-mentioned bipolar radiofrequency treatment device can realize a treatment mode of different treatment depths by changing the polarity of the electrodes of the first radio frequency electrode and the second radio frequency electrode of each bipolar radiofrequency treatment unit on the therapeutic surface, realizing precise treatment, and the treatment efficiency is extremely high.

In some embodiments, the above bipolar radiofrequency treatment device can achieve the adjustable depth based on the following principle. In different bipolar radiofrequency treatment devices, the RF energy generator can switch the two radio frequency electrode polarities, to form RF treatment current combinations of multi-channel different current directions. The use of different directions of radio frequency treatment currents can, due to their respective magnetic field directions generated by the currents, cause mutual attractions or rejections. Further, based on the RF electrodes in the treatment surface specific settings, subcutaneous current density and penetration can be self-regulated, thereby achieving subcutaneous radiofrequency energy penetration depth and energy aggregation control.

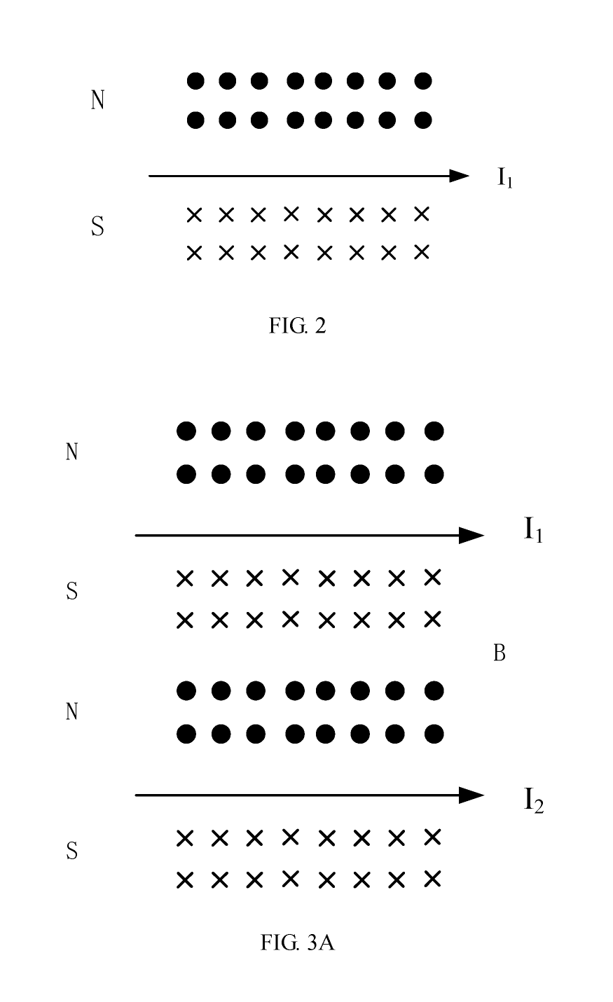

For example, in the case of only one set of bipolar radiofrequency treatment device, as shown in FIG. 2, a magnetic field is generated around the RF current I1 between the two RF electrodes, the current direction of the RF current I1 is from left to right, the magnetic field of the RF current I1 can have its direction determined from the Ampere rule, that the magnetic field above the RF current I1 is in the direction from the inside out, and below the RF current I1 the magnetic field direction is from outside toward inside. Similarly, if the RF current I1 current is from right to left, then the direction of the magnetic field of the RF current I1 is opposite to the aforementioned direction.

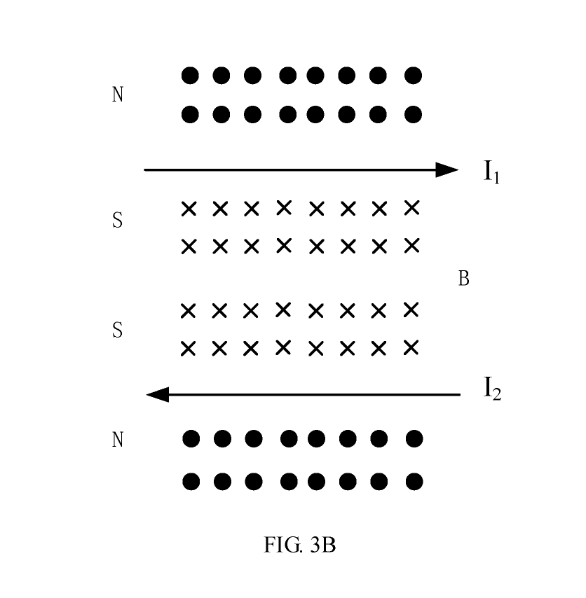

In another example, in the case of only two groups of bipolar radiofrequency treatment units, the radio frequency currents formed by the bipolar radiofrequency treatment units of each group are the RF current I1, and the RF current I2, respectively. As shown in FIG. 3A, if the RF current I1 and the RF current I2 are parallel and the current directions are the same, the electromagnetic fields formed by the RF current I1 and the RF current I2 are opposite in polarity in the intermediate region B, and the current density is made more intensified in the intermediate region B, resulting from the mutual attraction. As shown in FIG. 3B, the electromagnetic fields formed by the radio frequency current I1 and the radio frequency current I2 having opposite directions cause, in the middle region B, the repelling from each other, resulting in the current density in the middle region B becoming sparser.

In connection with the principles described above, several embodiments of the bipolar radiofrequency therapeutic device having a treatment depth self-regulating function are described in more detail below.

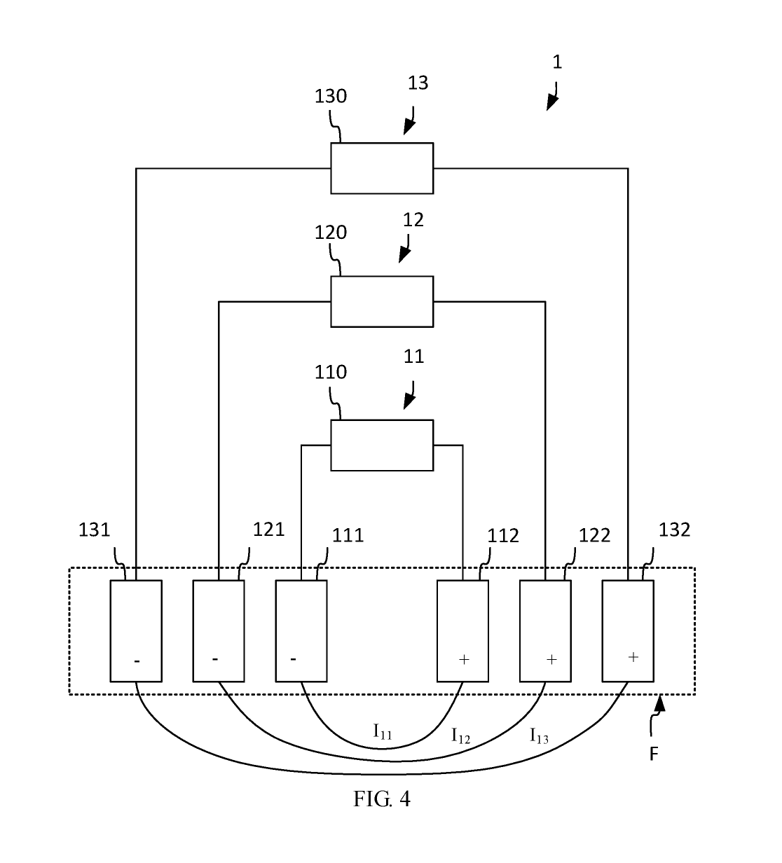

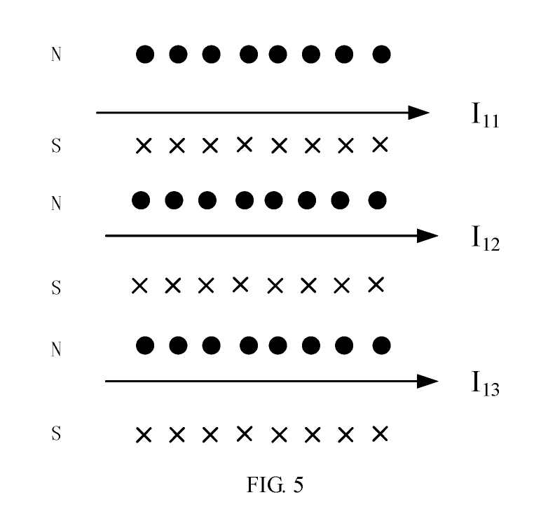

In some embodiments, as illustrated in FIG. 4, an apparatus is provided comprising three bipolar radiofrequency treatment devices or units, including: bipolar radiofrequency treatment device 11, bipolar radiofrequency treatment device 12, and bipolar radiofrequency treatment device 13, wherein bipolar radiofrequency treatment device 11 includes a radio frequency energy generator 110, and a radio frequency electrode 111 and a radio frequency electrode 112 connected to the RF energy generator 110. The bipolar radio frequency treatment device 12 includes a radio frequency energy generator 120, and a radio frequency electrode 121 and a radio frequency electrode 122 connected on the generator 120. The bipolar radio frequency treatment device 13 includes a radio frequency energy generator 130, and a radio frequency electrode 131 and a radio frequency electrode 132 connected to the radio frequency energy generator 130. In addition, the radio frequency electrode 111, the radio frequency electrode 121, and the radio frequency electrode 132 are spaced apart from each other on the therapeutic surface, and the radio frequency electrode 112, the radio frequency electrode 122, and the radio frequency electrode 132 are spaced apart on the other side of the therapeutic surface. Each radio frequency energy generator applies to two radio frequencies electrodes RF currents therebetween with the same direction, referring to FIG. 5. In this case, the magnetic fields generated between the neighboring RF currents have opposite directions, resulting in mutual attraction effects, and thus the RF current I11, the RF current I12, the RF current I13, become closer to each other. As a result, the skin current density in the subcutaneous area will increase. At the same time, based on the ohm law Q=I²Rt it can be derived that there is a focusing effect of energy, and heating will be more concentrated. Therefore, using this method, the current is more concentrated in the upper dermis, and targeted upper dermis heating can be realized.

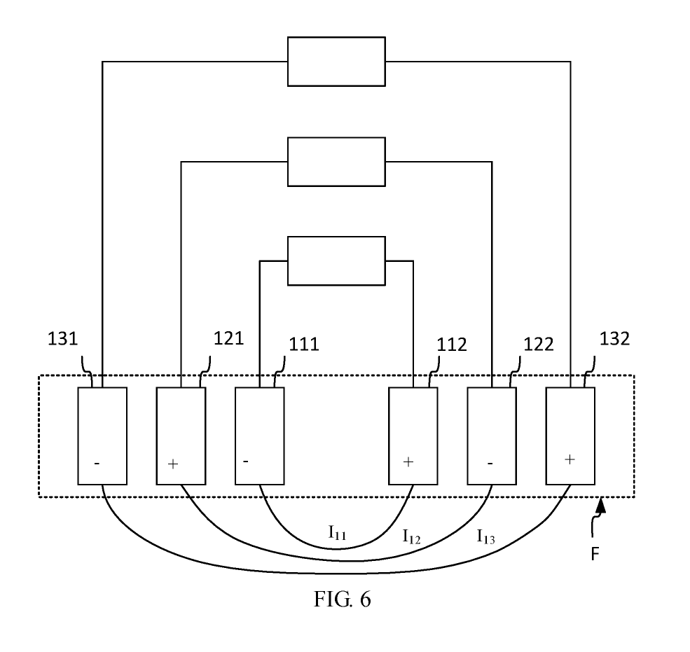

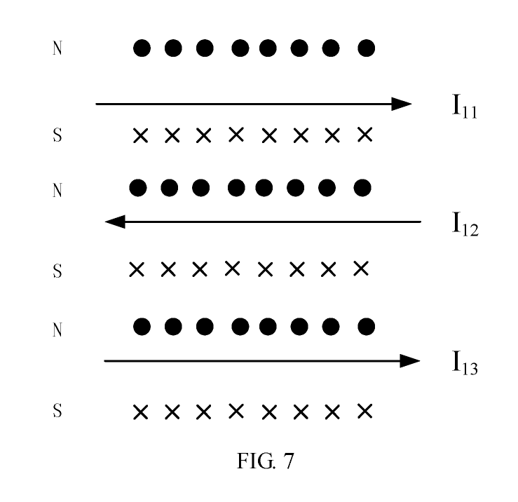

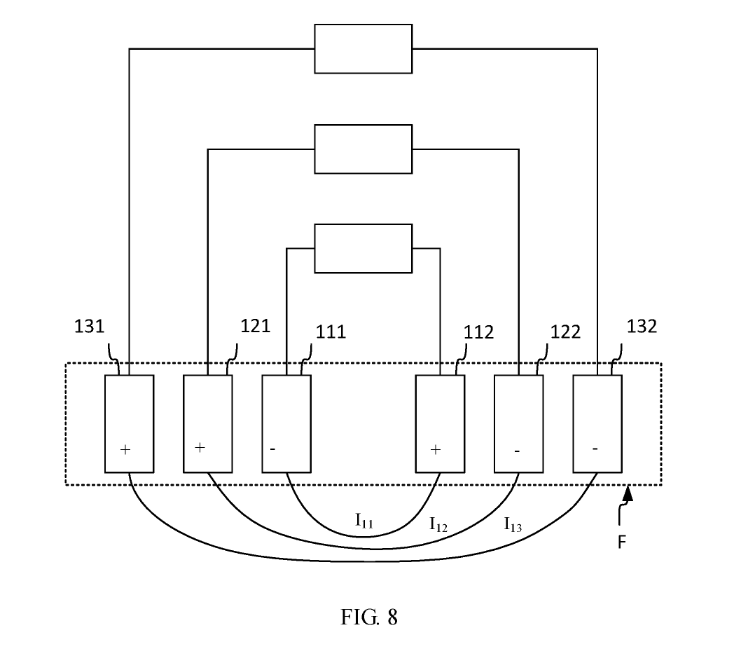

In some other embodiments, the configuration of the structure illustrated in FIG. 6 can be obtained by adjusting the polarity of the electrodes of the RF electrodes on one of the RF energy generators. As opposed to the previous embodiments: three bipolar radiofrequency treatment units or devices have RF currents with different directions underneath the treatment surface. The direction of the RF current I12 in the middle of the treatment surface is opposite to the RF current I11 and the RF current I13 located on both sides of the therapeutic surface. Referring to FIG. 7, the current direction of the RF current I12 is opposite to the direction of the RF current I11 and the radio frequency current I13, respectively, and the magnetic field directions of the magnetic field crossing region of the different radio frequency currents are the same, so that there is a. mutual repulsive effect, resulting in the RF current I12 And the RF current I11 and RF current I13 mutually exclusive. As such, the current density in the subcutaneous layers become sparse, and can be in the dermis layer, subcutaneous fat layer at all levels relatively evenly distributed. Therefore, this situation can promote the current penetration deeper, thereby facilitating fat reduction, shaping, facial relaxation and other treatment.

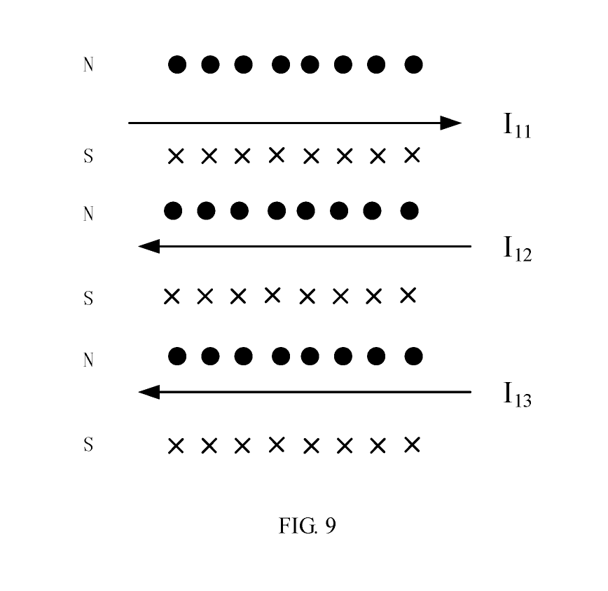

In some other embodiments, referring to FIG. 8, unlike the above two other embodiments, the RF current I11 is opposite to the current directions of the RF current I12 and the radio frequency current I13, respectively. Referring to FIG. 9, in this situation, the current density between the radio frequency current I11 and the radio frequency current I12 becomes sparse resulting from that the magnetic field directions are the same and have the repulsive effect in the magnetic field crossing region between the radio frequency current I11 and the radio frequency current I12, The current density between the radio-frequency current I12 and the radio-frequency current I13 becomes dense, resulting from that the magnetic fields have opposite directions in the magnetic field crossing region between the RF current I12 and the RF current I13. Therefore, subcutaneous energy penetration is deeper, can be mainly concentrated in the fat layer, suitable for fat-related improvement therapy, such as weight loss treatment.

In specific implementations, the polarities of the two RF electrodes on each bipolar radiofrequency treatment unit can be independently controlled by their respective RF energy generators. For example, at some point in time, the RF energy generator controls its associated first radio frequency electrode to be connected with the positive pole of the RF power source, and the second RF electrode is connected with the negative pole of the RF power source. At another time, the RF energy generator can control the first RF electrode to be connected with the negative pole of the RF power source, and the second RF electrode is connected with the positive pole of the RF power source. Therefore, the polarities between the RF electrodes can be switched. Such a control can be realized by software control or hardware control on the RF energy generator.

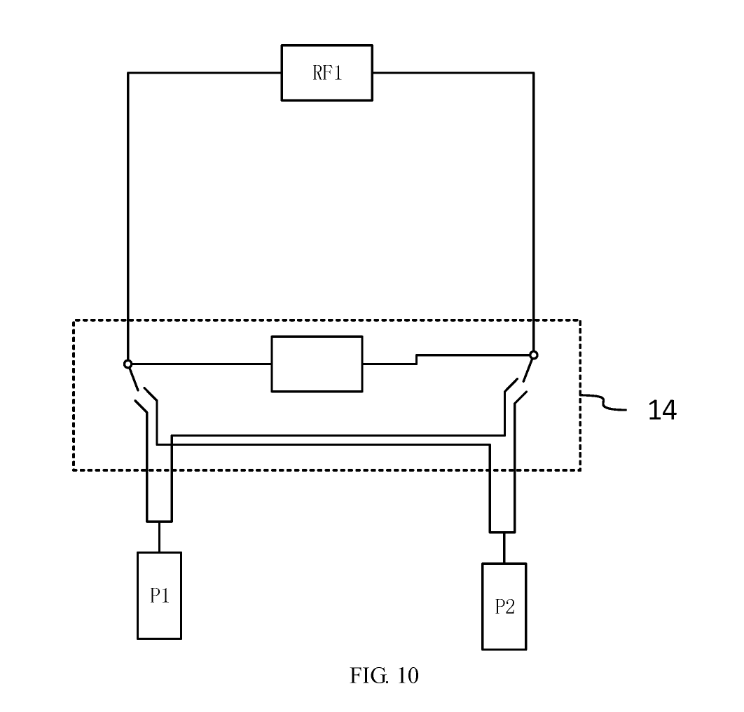

Of course, it is also possible to control the polarities of the two RF electrodes connected to the RF energy generator by means of a control mechanism connected to the RF energy generator. With reference to FIG. 10, for example, a combination of switching circuits can be included between the two radio frequency electrodes and the RF energy generator. The first RF electrode of the two RF electrodes can be connected to the positive electrode of the RF power source when the combination switch circuit is switched to a switch position, and the second radio frequency electrode is connected to the negative pole of the RF power source; and when the combined switch circuit is switched to another switching position, the first radio frequency electrode of the two radio frequency electrodes is connected to the negative electrode of the RF power source, the second RF electrode is connected to the positive side of the RF power supply. This way of connecting a control mechanism on a radio frequency energy generator can also realize control of the polarities of the RF electrodes connected to the RF power supply.

It should be noted that the above-described embodiments are exemplified by three groups of bipolar radiofrequency treatment units. In specific implementations, the number of bipolar radiofrequency treatment units may be 2, 3, 4, 5, . . . N, and can be selected according to specific needs.

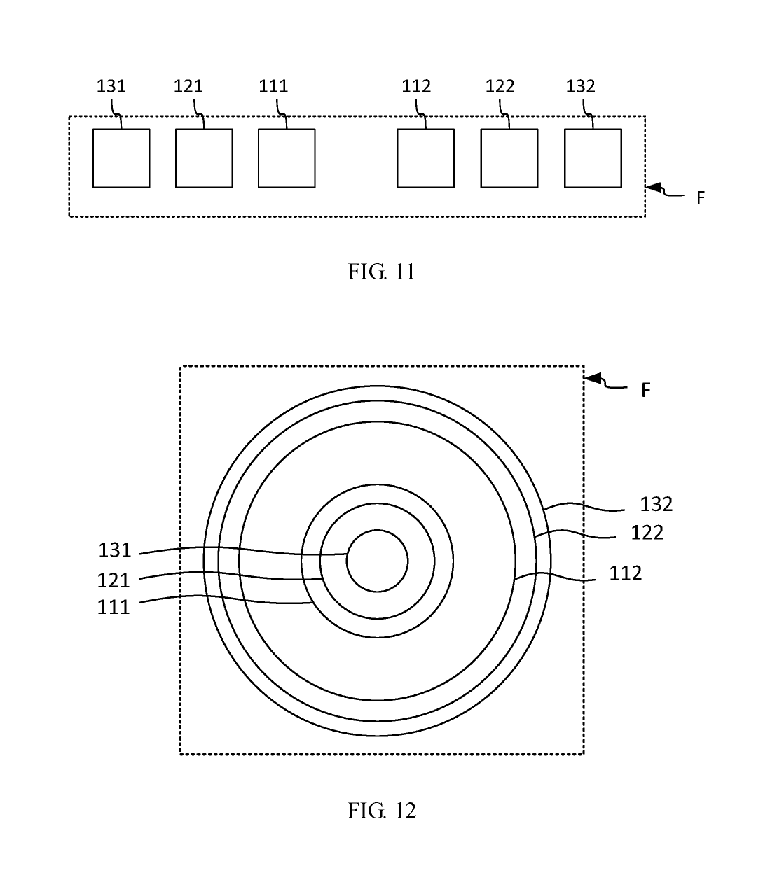

In some embodiments, as illustrated in FIG. 11, the shape of the first radio frequency electrode and the second radio frequency electrode of each bipolar radio frequency treatment unit may be a bulk radio frequency electrode, such as a square radio frequency electrode or a circular radio frequency electrode. In addition, as shown in FIG. 12, The shape of the first radio frequency electrode and the second radio frequency electrode may also be a ring-shaped radio frequency electrode, for example, a plurality of annular RF electrodes of different radii constituting a therapeutic surface of a concentric circular structure.

In some embodiments, the RF power generator can also be used to regulate the output power of the two RF electrodes connected to it. Generally, the output power of the RF energy generator is low, that is, it is generally controlled at or below 20 MHz, because there is generally no such problems for high frequencies as those described in the background.

In view of the above, by controlling the directions of the RF current through controlling the polarities of the two radio frequency electrodes on each bipolar radio frequency treatment unit, the sparseness and penetration depth of the RF current in the subcutaneous distribution can be improved, thereby realizing the radio frequency treatment depth self-regulation, in order to achieve targeted treatment of some symptoms, thereby enhancing the therapeutic effect and treatment efficiency. Therefore, various embodiments of the present disclosure effectively overcomes the shortcomings of the prior art and have a high degree of industrial use value.

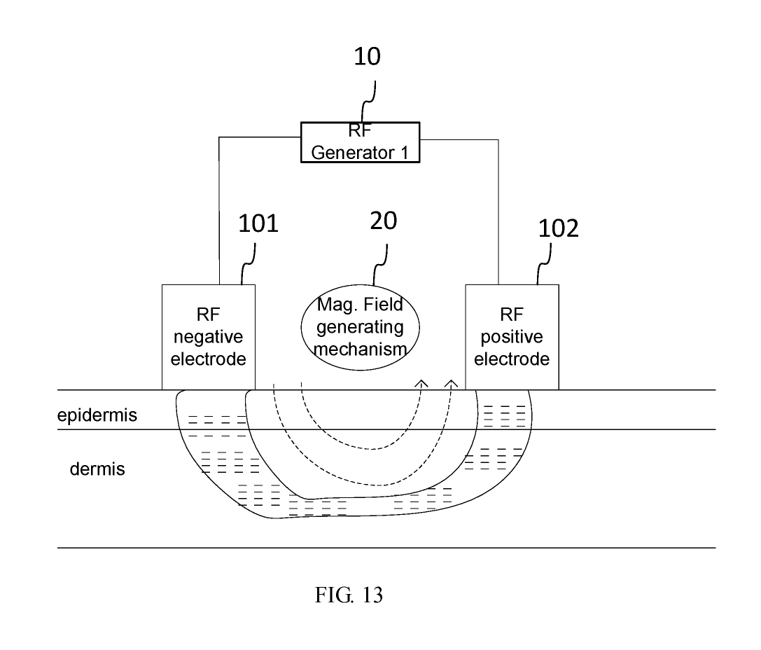

Referring to FIG. 13, some embodiments provide a bipolar radiofrequency treatment device that can adjust the depth of treatment, comprising: at least one or more radio frequency generators each formed by uniquely connecting a first radio frequency electrode and a second radio frequency electrode to form a set of bipolar radiofrequency treatment units, each of said first radio frequency electrodes and each of said second radio frequency electrodes being arranged in the same order and in reverse order, respectively, to form a therapeutic surface in the same direction, and in the same or/and different radio frequency generators at least one magnetic field generating mechanism is provided between the radio frequency electrodes or/and the second radio frequency electrodes.

The bipolar radiofrequency treatment device generates a magnetic field by providing a magnetic field between the current directions of the radio frequency electrodes. The magnetic field and that generated by the subcutaneous current itself can mutually affect each other, by the arrangement of the radio frequency electrode on the therapeutic surface, and use the effects of the magnetic fields being attract and mutually exclusive to improve the subcutaneous radiofrequency current density and aggregation, so as to control the subcutaneous radiofrequency energy penetration and aggregation, in order to achieve the precise treatment of the treatment area, thereby reducing the treatment time, and improving treatment efficiency.

In specific implementations, several embodiments can be implemented.

### Embodiment 1

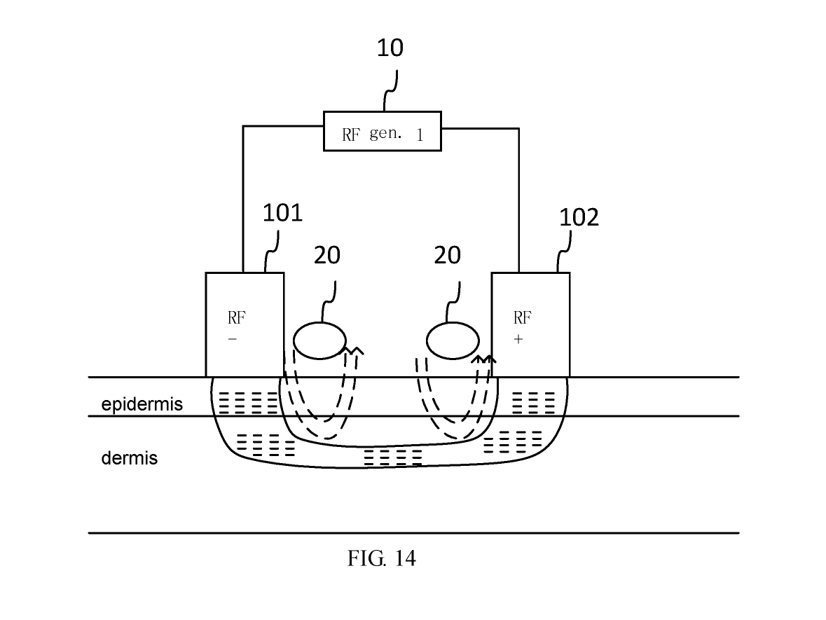

Only one set of bipolar radiofrequency treatment units are included, i.e., only one RF generator 10, as shown in FIGS. 13 and 14, are provided. One or more magnetic field generating structures or mechanisms are disposed between the first radio frequency electrode 101 and the second radio frequency electrode 102. If a plurality of magnetic field generating structures are employed, they are spaced apart from each other on the therapeutic surface, and coincides with the linear direction of the first radio frequency electrode and the second radio frequency electrode. The magnetic fields of different intensities and the different directions is generated by the one or more magnetic field generating structures so as to realize a variety of control of the radio frequency current between the first radio frequency electrode and the second radio frequency electrode.

### Embodiment 2

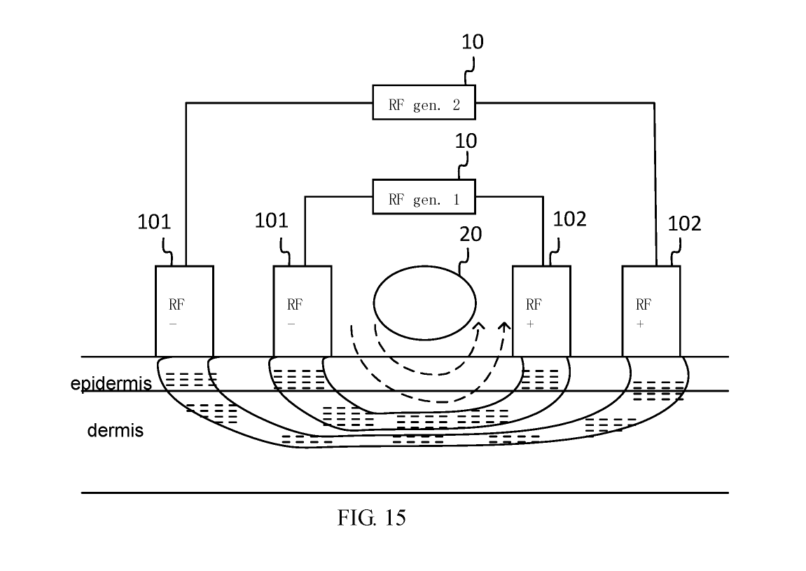

A plurality of sets of bipolar radiofrequency treatment units are included, i.e., having a plurality of radio frequency generators 10. As shown in FIG. 15, this solution is to provide a single or a plurality of magnetic field generating mechanisms only between a pair of radio frequency electrodes that are closest to each other, and to improve the treatment depth and density of subcutaneous radiofrequency currents in turn. In contrast to the above embodiment 1, this solution can enhance the degree of aggregation of the subcutaneous radio frequency current.

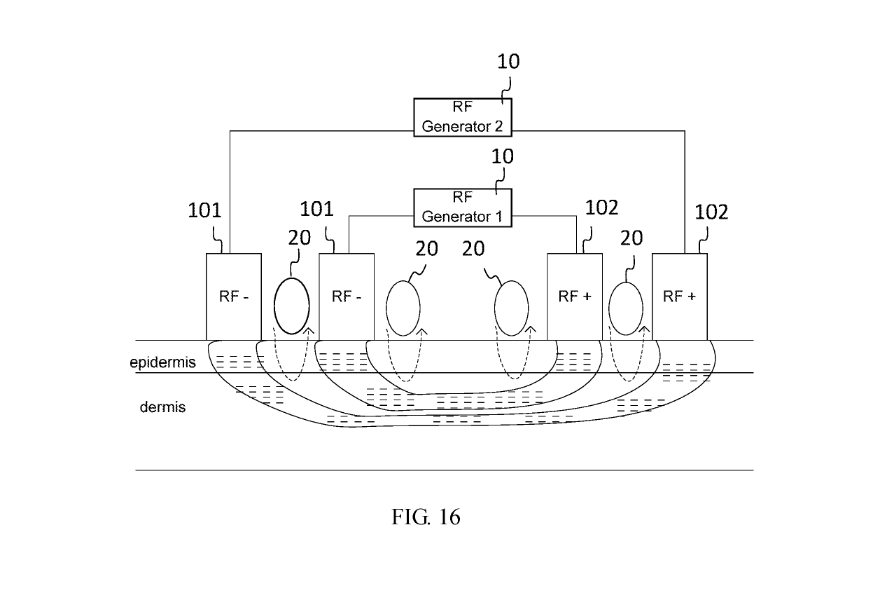

In addition, as shown in FIG. 16, it is also possible to provide a magnetic field generating mechanism between all or some of the RF electrodes, for example, between all of the first radio frequency electrodes 101 and/or the second radio frequency electrodes 102, or, of course, at least one magnetic field generating mechanism is provided between some of the first radio frequency electrodes 101 and/or the second radio frequency electrodes 102. In the figure, at least one magnetic field generating mechanism is provided between all the radio frequency electrodes so that the magnetic field generating mechanisms can be freely controlled according to the actual requirements, with a very high efficiency.

It is to be understood that the above views show only preferred embodiments, and other possible embodiments are also included within the scope of the embodiments, but only a detailed view is not given.

Further, in a specific embodiment, the magnetic field generating means may be implemented in a plurality of ways, for example, a magnet may be provided on the treatment surface, or may be implemented by using a battery coil.

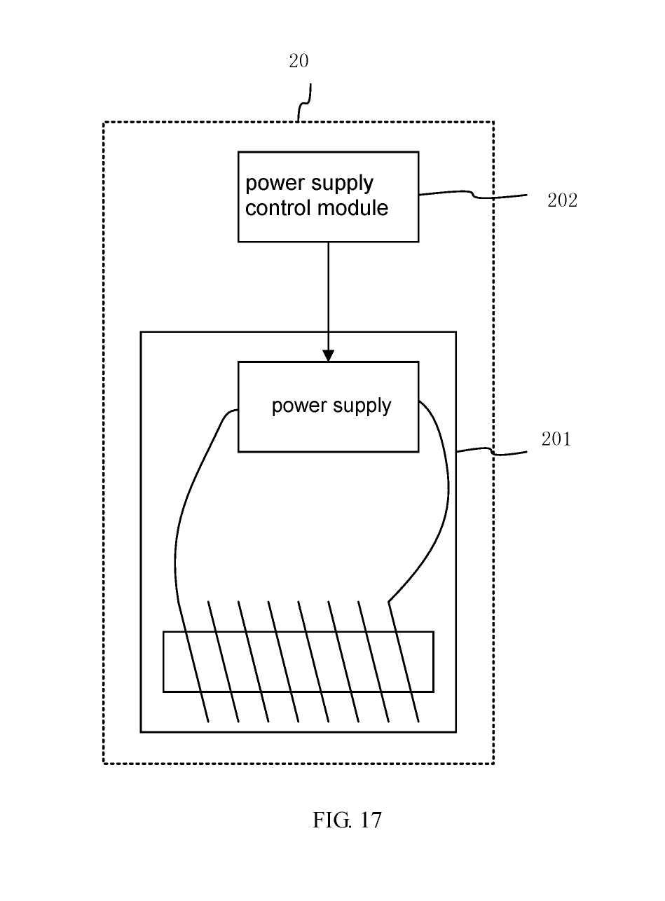

Specifically, an example in which an electromagnetic coil is used is described, with reference to FIG. 17. The magnetic field generating mechanism 20 may include an electromagnetic coil 201 and a power supply control module 202 for applying different current directions and/or different current amplitudes to the electromagnetic coil in accordance with a setting requirement. For example, the direction of the current in the electromagnetic coil can be adjusted by the power supply control module 202, and the direction of the magnetic field can be changed. Alternatively, the current intensity in the electromagnetic coil can be adjusted by the power supply control module 202, and the intensity of the magnetic field can be changed in turn. This control can be set according to specific needs.

It should be noted that the above embodiments have better effects on low-frequency RF generators.

In view of the above, various embodiments provide a magnetic field generating mechanism on the treatment surface to generate a magnetic field, which improves the density and the degree of aggregation of the subcutaneous radiofrequency current by utilizing the effects of mutual attraction and mutual repulsion of the magnetic fields, thereby controlling the penetration of the subcutaneous radiofrequency energy and aggregation, in order to achieve accurate treatment of the treatment area, thereby shortening the treatment time and improving the treatment efficiency.

All references cited herein are incorporated by reference in their entirety. Although specific embodiments have been described above in detail, the description is merely for purposes of illustration. It should be appreciated, therefore, that many aspects described above are not intended as required or essential elements unless explicitly stated otherwise. Various modifications of, and equivalent acts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. A bipolar radiofrequency (RF) treatment apparatus with an adjustable treatment depth, the apparatus comprising:
a plurality of bipolar RF treatment devices each including an RF energy generator, and a first RF electrode and a second RF electrode uniquely connected to the RF energy generator, wherein:
the first and second RF electrodes are arranged on a same treatment surface in a same order and in a reverse order, respectively; and
each of the RF energy generators is configured to switch polarities of the first RF electrode and the second RF electrode connected thereto.

2. The apparatus of claim 1, wherein the first RF electrodes have a first equal interval on the treatment surface, and the second RF electrodes have a second equal interval on the treatment surface.

3. The apparatus of claim 1, the first and second RF electrodes each have a rectangular shape.

4. The apparatus of claim 1, the first and second RF electrodes each have an annular shape.

5. The apparatus of claim 1, wherein each of the RF energy generators is configured to control a current frequency of the first and second RF electrodes connected thereto.

6. The apparatus of claim 1, wherein each of the RF energy generators is configured to apply a current frequency of no more than 20 MHz to the first and second RF electrodes connected thereto.

7. The apparatus of claim 1, further comprising at least one magnetic field generating mechanism disposed between two of the first and second RF electrodes.

8. The apparatus of claim 7, wherein the apparatus comprises one RF energy generator, and at least one magnetic field generating mechanism is disposed at the treatment surface between the first and second RF electrodes connected to the RF energy generator.

9. The apparatus of claim 7, wherein the apparatus comprises a plurality of RF energy generators, and at least one magnetic field generating mechanism is disposed at the treatment surface between two of the first and second RF electrodes.

10. The apparatus of claim 9, wherein the plurality of magnetic field generating mechanisms are distributed on the treatment surface.

11. The apparatus of claim 7, wherein the magnetic field generating mechanism comprises a coil, and a power supply control module configured to apply electrical currents of different directions or/and different amplitudes to the coil.

12. The apparatus of claim 7, wherein the magnetic field generating mechanism comprises a magnet.

13. The apparatus of claim 7, wherein the RF energy generator is a low-frequency RF energy generator.

14. The apparatus of claim 7, wherein the first RF electrodes have a first equal interval on the treatment surface, and the second RF electrodes have a second equal interval on the treatment surface.

15. The apparatus of claim 7, wherein each of the first and second RF electrodes has a rectangular shape.

16. The apparatus of claim 7, wherein each of the first and second RF electrodes has an annular shape.

17. A bipolar radiofrequency (RF) treatment apparatus comprising:
a plurality of bipolar RF treatment devices each including an RF energy generator, and a first RF electrode and a second RF electrode uniquely connected to the RF energy generator, wherein:
the first and second RF electrodes are arranged on a same treatment surface in a same order and in a reverse order, respectively;
each of the RF energy generators is configured to switch polarities of the first RF electrode and the second RF electrode connected thereto; and
the apparatus is configured to adjust a treatment depth by controlling mutual attraction and/or repulsion between magnetic fields associated with electrical currents between the first and second RF electrodes.

18. The apparatus of claim 17, further comprising a magnetic field generating mechanism configured to further affect a distribution of the electric currents.

19. A method of skin treatment using a bipolar radiofrequency (RF) treatment apparatus, wherein the apparatus comprises:
a plurality of bipolar RF treatment devices each including an RF energy generator, and a first RF electrode and a second RF electrode uniquely connected to the RF energy generator,
wherein the first and second RF electrodes are arranged on a same treatment surface in a same order and in a reverse order, respectively;
the method comprising:
switching, using each the RF energy generators, polarities of the first RF electrode and the second RF electrode connected to the RF energy generator, to adjust a distribution of electrical currents between the first and second RF electrodes based on mutual attraction and/or repulsion between magnetic fields associated with the electrical currents, thereby realizing control of a treatment depth and treatment current density.

19. The method of claim 19, further comprising further adjusting the distribution of electrical currents using a magnetic field generating mechanism disposed between the first and second RF electrodes.
